(19) United States
(12) Patent Application Publication
(10) Pub. No.: US 2012/0209306 A1
(43) Pub. Date: Aug. 16, 2012

(54) ELASTOMERIC TROCAR SAFETY SHIELD

(75) Inventor: Gregory Okoniewski, North Haven, CT (US)

(73) Assignee: Tyco Healthcare Group LP

(21) Appl. No.: 13/360,026

(22) Filed: Jan. 27, 2012

Related U.S. Application Data
(60) Provisional application No. 61/442,856, filed on Feb. 15, 2011.

(51) Int. Cl. A61B 17/34

(52) U.S. Cl. 606/185

(57) ABSTRACT

A trocar is presented including a cannula having a proximal end and a distal end, the proximal end of the cannula connected to a cannula assembly. The trocar further includes an obturator having a penetrating tip, the obturator extending through at least a portion of the cannula and adaptable for longitudinal movement relative to the cannula. Also, a safety shield is provided being fixedly secured in a non-adjustable configuration to the distal end of the cannula, the penetrating tip of the obturator creating a slit through the safety shield for penetrating tissue once an actuation mechanism has been activated.

### CROSS REFERENCE TO RELATED APPLICATION

The present application claims the benefit of and priority to U.S. Provisional Application Ser. No. 61/442,856, filed on Feb. 15, 2011, the entire contents of which are incorporated herein by reference.

### BACKGROUND

1. Technical Field

The present disclosure relates to a surgical trocar used to puncture tissue and, more particularly, to a trocar having a non-adjustable elastomeric safety shield fixedly secured to a distal end of a cannula of the trocar.

2. Background of Related Art

A trocar generally comprises two main components, a trocar tube and an obturator having an obturator tip. The trocar tube may be inserted through the skin to access a body cavity through the tube in which laparoscopic or arthroscopic surgery may be to be performed. In order to penetrate the skin, the distal end of the trocar tube may be placed against the skin and an obturator may be inserted through the tube. By pressing against the proximal end of the obturator, the obturator tip may be forced through the skin until it enters the body cavity. At this time, the trocar tube may be inserted through the perforation made by the obturator and the obturator may be withdrawn, thus leaving the trocar tube as an accessway to the body cavity.

A significant force may be required to cause the obturator tip to penetrate the skin and underlying tissue. When the obturator tip breaks through this tissue, resistance to penetration may be suddenly removed, and the obturator tip may suddenly penetrate to reach internal organs of the body, which may cause lacerations and other injury to the internal organs. To avert this danger to the patient, trocars have been developed which carry a spring-loaded tubular shield within the trocar tube and surrounding the obturator, as well as the obturator tip. However, such spring-loaded mechanisms associated with safety shields of trocars used for averting danger to the patient are often cumbersome and difficult to use.

### SUMMARY

In accordance with the present disclosure, a trocar is provided. The trocar includes a cannula having a proximal end and a distal end, the proximal end of the cannula connected to a cannula assembly. The trocar further includes an obturator having a penetrating tip, the obturator extending through at least a portion of the cannula and adaptable for longitudinal movement relative to the cannula. Also, a safety shield is provided being fixedly secured in a non-adjustable configuration to the distal end of the cannula, the penetrating tip of the obturator creating a slit through the safety shield for penetrating tissue once an actuation mechanism has been activated.

### BRIEF DESCRIPTION OF THE DRAWINGS

Various embodiments of the presently disclosed trocar assembly are described hereinbelow with references to the drawings, wherein:

FIG. 1 is a side view of a trocar having a safety shield, where an obturator is positioned in a retracted, non-deployed position, in accordance with the present disclosure;

FIG. 2 is a side view of a distal end of the trocar having the safety shield of FIG. 1, where the obturator is positioned in a retracted position, in accordance with the present disclosure;

FIG. 3 is a side view of the distal end of the trocar having the safety shield of FIG. 1, where the obturator is positioned in a retracted, yet semi-deployed position, in accordance with the present disclosure;

FIG. 4 is a side view of the distal end of the trocar having the safety shield of FIG. 1, where the obturator is positioned in an expanded, fully deployed position, in accordance with the present disclosure; and

FIG. 5 is a side view of the distal end of the trocar having the safety shield of FIG. 1, where the obturator is positioned in a retracted position after piercing the safety shield, in accordance with the present disclosure.

Further scope of applicability of the present disclosure will become apparent from the detailed description given hereinafter. However, it should be understood that the detailed description and specific examples, while indicating preferred embodiments of the present disclosure, are given by way of illustration only, since various changes and modifications within the spirit and scope of the present disclosure will become apparent to those skilled in the art from this detailed description.

### DETAILED DESCRIPTION OF THE EMBODIMENTS

In the figures and in the description that follows, in which like reference numerals identify similar or identical elements, the term “proximal” will refer to the end of the apparatus which is closest to the operator during use, while the term “distal” will refer to the end which is farthest from the operator, as is traditional.

Prior to describing the present disclosure in further detail, it will first be helpful to define various terms that will be used throughout the following discussion. For example:

The term “connect” or “connecting” may refer to adhere, affix, anchor, attach, band, bind, bolt, bond, brace, button, cohere, fasten, couple, embed, establish, fix, grip, hold, hook, implant, link, lock, lodge, screw, seal, rivet, tack on, tighten, or unite. The term “connect” or “connecting” may refer to linking/fastening/attaching/locking any type of materials or elements or components or units in a removable/detachable/interchangeable manner.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

Embodiments will be described below while referencing the accompanying figures. The accompanying figures are merely examples and are not intended to limit the scope of the present disclosure.

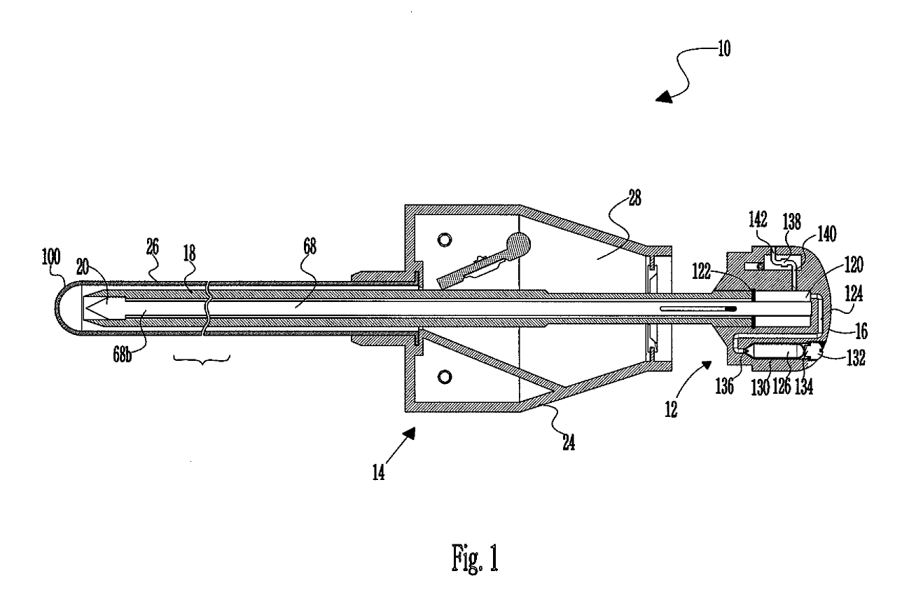

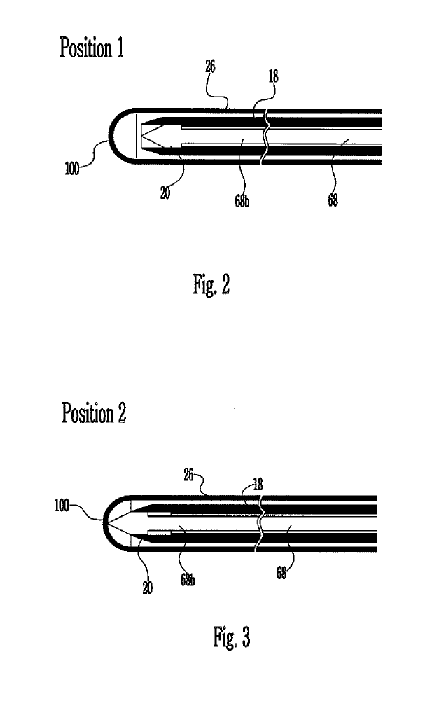

Referring to FIGS. 1-5, a trocar 10 includes an obturator assembly 12 and a cannula assembly 14. The obturator assembly 12 includes obturator housing 16, obturator shaft 18, and obturator tip 20. Cannula assembly 14 includes cannula housing 24 and cannula sleeve 26 which may be secured to the cannula housing 24 and may extend outwardly therefrom. Cannula housing 24 may be configured and dimensioned to interfit with obturator housing 16, so that obturator shaft 18 may slide within cannula sleeve 26 when the two assemblies are interfitted. Preferably, cannula housing 24 has an open interior for mounting valve system 28. The valve system 28 may be provided to maintain a gas tight seal within the cannula housing 24.

The indicator portion of trocar 10 will now be described with reference to FIGS. 1-5. Generally, the indicator portion of the trocar assembly 10 of the present disclosure may be responsive to the flow of gas through obturator assembly 12 and may provide a surgeon with a visual indication that the obturator assembly 12 has penetrated body tissue and entered a body cavity. Channel 68 may extend through obturator shaft 18, and may have a distal end 68b aligned with obturator shaft 18.

The proximal end of obturator shaft 18 is slidably positioned within biasing chamber 120. Seal member 122 is secured to the proximal end of obturator shaft 18 and is provided to maintain a gas tight seal within the chamber. In this exemplary embodiment, the gas pressure source is a CO₂ cartridge 126 positioned within obturator housing 16. Cartridge 126 is secured within cartridge chamber 130 by threaded cover 132. Preferably, cover 132 may include resilient member 134 which engages cartridge 126 and maintains the gas pressure within cartridge chamber 130. Pin 136 may be provided to puncture cartridge 126 when cover 132 is threaded into chamber 130 and gas from cartridge 126 is passed to biasing chamber 120 via channel 124. Relief valve 138 may be operatively connected to chamber 120 via channel 140 and has relief vent 142 passing through obturator housing 128.

In operation, gas pressure from cartridge 126 may move obturator shaft 18 to the protective position, as shown in FIG. 1. Relief valve may be closed to maintain the gas pressure. When penetrating the body tissue, obturator shaft 18 may move proximally within chamber 120. Proximal movement of the tube may cause the gas pressure within the chamber 120 to increase. In some instances the pressure within the chamber may increase to a level where obturator shaft 18 is prevented from further proximal movement. To compensate for the increased pressure, relief valve 138 may open when the pressure within the chamber reaches a predetermined level, thus allowing further proximal movement of the tube.

When the body tissue is penetrated, the back-pressure within the chamber caused by obturator shaft 18 ceases. Relief valve 138 then closes and cartridge 126 increases the pressure within chamber 120 to move obturator shaft 18 distally to the protective position.

Additionally, the obturator includes a safety shield 100. In operation, the channel 68 of the obturator shaft 18 may be slidably and removably located within the cannula sleeve 26 and may be inserted into the cannula assembly 14 by way of an opening (not shown). The trocar 10 may be used to puncture a hole in tissue by placing the distal end of the cannula sleeve 26 against the tissue, and activating an actuation mechanism. The longitudinal movement of the obturator tip 20 may be enabled through an actuating mechanism located within the cannula assembly 14 and/or the obturator housing 16. The actuating mechanism may be any type of mechanism contemplated by one skilled in the art, such as, but not limited to, a spring-loaded mechanism. The actuating mechanism may be a manual mechanism or an automated mechanism.

As the actuation mechanism located within the cannula assembly 14 and/or the obturator housing 16 may be activated, the cannula assembly 14 may begin to shift the obturator shaft 18 to move the channel 68 and the obturator tip 20 toward the inner portion of the safety shield 100. The cannula sleeve 26 and the safety shield 100 do not retract or move as the actuation mechanism may be activated. The component that may be movable or adjustable may be the obturator tip 20 connected to the obturator shaft 18. This movement or adjustment to the obturator tip 20 located within the fixed, non-movable cannula sleeve 26 exposes obturator tip 20, which punctures the tissue. Thus, the safety shield 100 may be fixedly secured in a non-adjustable configuration to the distal end of the cannula sleeve 26. Additionally, the cannula sleeve 26 and the safety shield 100 may be constructed as a single, continuous, and non-adjustable unit in order to only allow longitudinal movement of the obturator tip 20.

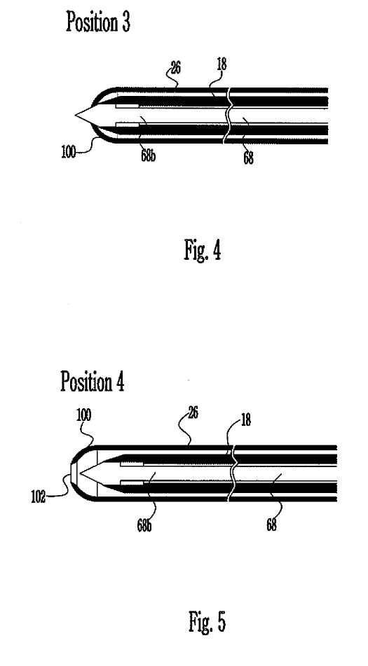

FIG. 3 illustrates the actuation mechanism activated in order to shift or move the obturator tip 20 so that the obturator tip 20 contacts the inner surface of the safety shield 100. FIG. 4 illustrates the actuation mechanism activated even further in order to shift or move the obturator tip 20 so that the obturator tip 20 breaks through the safety shield 100 and may be in a fully deployed or exposed position in order to puncture the tissue. The breakage of the safety shield 100 creates a slit 102, as shown in FIG. 5. FIG. 5 illustrates the obturator tip 20 retracting to its initial position after being actuated by the actuating mechanism located in either the cannula assembly 14 or the obturator housing 16. Once the obturator tip 20 retracts to its initial position, the safety shield 100 includes a slit 102.

The obturator tip 20 may be a substantially triangular surface having an apex. However, one skilled in the art may contemplate a plurality of different shaped and sized obturators for perforating tissue or any other medium. Additionally, the safety shield 100 may be configured as a rounded distal end or as a nose cone. The rounded distal end may include a biocompatible lubricated surface for lubricating the tissue it comes into contact with the obturator tip 20. The rounded distal end may further include a semi-rigid elastomeric covering. Of course, it is contemplated that the safety shield 100 may be of any shape or size in accordance with desired applications. The safety shield 100 may be constructed from any type of suitable material for permitting the obturator tip 20 to pierce through the safety shield 100.

Additionally, in accordance with the example embodiments, the safety shield 100 initially covers or encloses the obturator tip 20. When the obturator tip 20 is pressed into tissue via an actuating mechanism located within the cannula assembly 14 or the obturator housing 16, that applied pressure or force causes the obturator tip 20 to first cut through the safety shield 100, thereby uncovering the obturator tip 20, so that the obturator tip 20 may then cut through the tissue. Once through the tissue, the semi-rigidity of the safety shield 100 enables it to return to its original shape (albeit a cut through it, such as slit 102), which substantially covers the obturator tip 20 once again. Thus, the safety shied 100 is not spring loaded, but rather fixedly secured to and extends from a distal end of the cannula sleeve 26. In other words, the cannula sleeve 26 and the safety shield 100 may be constructed as a single, continuous, and non-adjustable unit. Stated in a different way, the safety shield 100 may be incorporated into the trocar assembly 10 in a fixed and non-movable manner.

In an alternate embodiment, the cannula sleeve 26 and the safety shield 100 may be separate and distinct units that are fixedly secured to each other in a non-adjustable configuration. Stated in a different way, the safety shield 100 may be configured as a removable unit for enabling attachable and detachment of a plurality of different safety shields to the cannula sleeve 26. However, in operation, the cannula sleeve 26 and the safety shield 100 remain in a fixed, non-adjustable configuration once the components are put together into any type of trocar assembly. Once again, the only movable components within the trocar 10 may be the obturator tip 20 and any components/elements/units used to aid the obturator tip 20 in longitudinal movement with respect to the cannula sleeve 26. It is contemplated that such longitudinal movement of the obturator tip 20 may be actuated by any type of mechanism located within the cannula assembly 14 and/or the obturator housing 16.

In summary, the safety shield 100 may cover the obturator tip 20 until the trocar 10 is ready for use. When ready to use, the obturator tip 20 may be inserted through the cannula assembly 14 via the channel 68. The actuating mechanism may then be moved distally, thus locking the obturator tip 20 in an exposed position. The trocar 10 may optionally remain locked in a retracted position until a force has been applied to release the retracting mechanism. For example a force may be applied via an actuation mechanism within the cannula assembly 14 and/or the obturator housing 16 to push the obturator tip 20 via the obturator shaft 18 through the abdominal wall of a patient. When the obturator tip 20 has pierced the abdominal wall, the distal end of the safety shield 100 may break, the force on the obturator tip 20 may be released, and the obturator tip 20 may then retract into the safety shield 100. Thus, the obturator tip 20 may puncture the safety shield 100 to create a slit 102. Of course, the slit 102 of the safety shield 100 remains broken for further uses of the trocar 10. However, it is contemplated that the safety shield 100 itself may be removed and replaced after a number of uses. In such operations, the safety shield 100 and the cannula sleeve 26 remain in a fixed, non-movable relationship with respect to each other.

It will be understood that there are to be no limitations as to the dimensions and shape of the trocar 10 and/or safety shield 100, including the obturator tip 20, or the materials from which the trocar 10 and/or safety shield 100 is manufactured or the electronics that may be used to run such a trocar assembly 10. It is to be realized that the optimum dimensional relationships for the parts of the present disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

From the foregoing and with reference to the various figure drawings, those skilled in the art will appreciate that certain modifications can also be made to the present disclosure without departing from the scope of the same, While several embodiments of the disclosure have been shown in the drawings, it is not intended that the disclosure be limited thereto, as it is intended that the disclosure be as broad in scope as the art will allow and that the specification be read likewise, therefore, the above description should not be construed as limiting, but merely as exemplifications of particular embodiments. Those skilled in the art will envision other modifications within the scope and spirit of the claims appended hereto.

What is claimed is:

1. A trocar comprising:
a cannula having a proximal end and a distal end, the proximal end of the cannula connected to a cannula assembly;
an obturator having a penetrating tip, the obturator extending through at least a portion of the cannula and adaptable for longitudinal movement relative to the cannula; and
a safety shield fixedly secured in a non-adjustable configuration to the distal end of the cannula, the penetrating tip of the obturator creating a slit through the safety shield for penetrating tissue once an actuation mechanism has been activated.

2. The trocar according to claim 1, wherein the obturator includes an obturator shaft fixed relative to an obturator housing.

3. The trocar according to claim 1, wherein the penetrating tip of the obturator is a substantially triangular surface having an apex.

4. The trocar according to claim 1, wherein the safety shield is configured as a rounded distal end.

5. The trocar according to claim 4, wherein the rounded distal end includes a biocompatible lubricated surface.

6. The trocar according to claim 4, wherein the rounded distal end is a semi-rigid elastomeric covering.

7. The trocar according to claim 1, wherein the safety shield is adapted to extend to an extended position upon penetration of the tissue and is adapted to retract in a retracted position after piercing of the tissue.

8. The trocar according to claim 1, wherein the cannula and the safety shield are constructed as a single, continuous, and non-adjustable unit.

9. The trocar according to claim 1, wherein the cannula and the safety shield are separate and distinct units that are fixedly secured to each other in a non-adjustable configuration.

10. The trocar according to claim 1, wherein the safety shield is configured as a removable unit for enabling attachable and detachment of a plurality of different safety shields to the cannula.
